(19) United States
(12) Patent Application Publication
(10) Pub. No.: US 2021/0275747 A1
(43) Pub. Date: Sep. 9, 2021

(54) DEVICE HAVING SYRINGE DISPENSERS FOR ADMINISTERING THERAPEUTIC SUBSTANCES USING A HIGH VELOCITY LIQUID-GAS STREAM

(71) Applicant: TAV - TECH LTD, Yehud (IL)

(72) Inventors: Ronen SOBEL, Kochav Ya'ir (IL); Alexander POLYAKOV, Qidmat Tsvi (IL)

(73) Assignee: TAV - TECH LTD, Yehud (IL)

(21) Appl. No.: 17/260,251

(22) PCT Filed: Aug. 25, 2019
(86) PCT No.: PCT/IL2019/050952

§ 371 Date: Jan. 14, 2021

Related U.S. Application Data
(60) Provisional application No. 62/730,011, filed on Sep. 12, 2018.

(51) Int. Cl. A61M 5/30; A61M 5/24

(52) U.S. Cl. CPC A61M 5/3007; A61M 2005/3128; A61M 5/24

(57) ABSTRACT

Disclosed herein is a device for administering a liquid therapeutic substance to tissue, including a housing having a liquid inlet port a gas inlet port connected to a pressurized gas source; at least one therapeutic substance supply assembly mounted onto said housing, each therapeutic substance supply assembly including at least one syringe dispenser connector configured for receiving a syringe dispenser containing a predefined quantity or concentration of the liquid therapeutic substance, each syringe dispenser in fluid flow communication with one of at least one valve projecting from and external to said assembly, said syringe dispenser includes a container configured to store the liquid therapeutic substance and a piston configured to be pulled into the container as the liquid therapeutic substance is removed from said container under suction so as to flow through said at least one valve.

### FIELD OF INVENTION

The invention relates to the field of generally, to devices for administering therapeutic substances in predefined dosages and concentrations, and, more specifically, to devices for applying a high velocity therapeutic liquid-gas stream for administering such substances to body tissue in predefined dosages and concentrations.

### BACKGROUND

Devices for dermal abrasion of exposed in vivo tissue are known in the art. One such device is described in International Publication Number WO 2005/065032, “A High Velocity Liquid-Gas Mist Tissue Abrasion Device”, included herein by reference. This document also provides a general overview of the prior art of dermal abrasion and dermal abrasion devices.

Disclosed in the above referenced document is a device for dermal abrasion employing a high-velocity liquid-gas streaming mist. The disclosed device is particularly successful in overcoming the difficulty of stagnant boundary layers. When a fluid stream is employed to irrigate a tissue surface, a boundary layer is formed which is characterized by having a fluid velocity which decreases sharply adjacent to the flow surface, being virtually zero at the tissue surface. As a result, particles which are smaller than the thickness of the boundary layer of the fluid stream are often difficult or impossible to remove. The smallest particles in the boundary layer exhibit a drag resistance of a magnitude sufficient for these particles to remain attached to the surface and to resist being swept away by the fluid stream. The device disclosed in the above referenced document overcomes this difficulty, its liquid-gas streaming mist producing a boundary layer of minimal to negligible thickness.

However, neither the device disclosed in the above-mentioned document nor other prior art devices discussed therein provides for easy treatment of abraded tissue with therapeutic substances in predefined dosages and/or concentrations. Additionally, the above-mentioned device and other prior art devices require relatively large liquid and gas sources, suitable for use with a plurality of patients. These sources are positioned distant from the device necessitating the use of connecting tubes which inter alia impede use, especially one-hand use, of the devices.

### DETAILED DESCRIPTION OF THE PRIOR ART

The liquid-gas stream consists of one or more therapeutic liquids provided at a high velocity, generally within the range of sub-sonic to super-sonic. To achieve these high velocities, gas is discharged from a device containing a stream jet nozzle arrangement, the arrangement containing one or more converging-diverging gas nozzles configured to accelerate the flow of gas so as to discharge it at an elevated velocity. A low rate of flow of therapeutic liquid is discharged into the elevated velocity flow of gas, thereby accelerating the discharged therapeutic liquid as a therapeutic stream of accelerated droplets. The volumetric rate of flow of therapeutic liquid from the device is relatively low, thereby essentially preventing the formation of a virtually stagnant liquid boundary layer on the surface of the tissue to which the therapeutic substances is being administered.

The housing of the device is in fluid flow communication with one or more containers containing one or more therapeutic substances. The therapeutic substances may be provided in bottles, vials, ampoules, or any other suitable containers. The containers are removably affixed to and positioned on the housing via a therapeutic substance supply assembly as described below and shown in FIGS. 8A-8G. The containers containing the therapeutic substances are generally single-use containers which contain predefined quantities and/or concentrations of therapeutic substances.

When the therapeutic liquid administered by the present invention is saline solution, the invention can be employed to clean a tissue surface. Subsequently, additional therapeutic substances, such as medications, nutrients, moisturizers or colorants may be administered. These therapeutic substances may be in liquid, emulsion or soluble powder form. This allows for more efficient dosing of the therapeutic substances, since, as will be appreciated by persons skilled in the art, the substances removed by cleaning would, if left in place, likely impede application and/or absorption of the desired therapeutic substances to the tissue undergoing therapeutic treatment.

The therapeutic substance supply assembly attached to the substantially tubular shaped housing of the present invention may include control valves operative for introducing into the device of the present invention a mixed flow of saline solution and other therapeutic substances. The valves can be used to obtain a desired concentration therein which can further be controlled, typically but without limiting the invention, by the operator during operation, to produce the mixed flow at specified times and for specified intervals. The device of the present invention would then accordingly produce a mixed therapeutic stream as desired and needed. Thus, as described above, a tissue surface could first be cleaned by saline solution and then dosed therapeutically with a medication solution when it is ready to optimally receive the dosage.

In an alternative embodiment of the present invention, instead of one mixed flow as mentioned hereinabove, the device of the present invention may be controlled and used to produce a number of therapeutic liquid flows for discharge into the elevated velocity gas flow. The therapeutic substances may also be turned on and off at specified times and for specified intervals. This arrangement also produces a mixed therapeutic stream as desired and needed. For example, the present invention can be used to treat a human scalp even where hair is present. First, the device provides an accelerated saline stream to clean the scalp of extraneous material, excess oils, and dead sloughed off epidermal tissue such as is known to produce dandruff. Then a moisturizing, nutrient, anti-dandruff, or anti-hair loss therapeutic substance is included in the accelerated stream to apply the desired therapeutic treatment to the scalp.

It should further be noted that the present invention is capable of applying the therapeutic substance to the desired tissue both topically and subcutaneously. Investigations employing prototype versions of the present invention have shown that the accelerated therapeutic stream produced thereby will, for suitable droplet flow velocities and time of exposure of the tissue to the droplet flow, penetrate the tissue surface. This capacity of non-invasive subcutaneous treatment and dosage is a further advantage of the present invention.

It is contemplated that the present invention can also be used in lavage of hollow organs of the body.

The discussion in conjunction with FIGS. 1-7 which follows is directed to an exemplary prior art stream jet delivery nozzle arrangement for accelerating a liquid/gas stream in the device of the present invention. In addition to the stream jet delivery nozzle arrangement shown in FIGS. 1-7, other jet delivery nozzle arrangements known in the art may also be used. The housing and control elements described and shown in FIGS. 1-7 are not the housing and control elements envisioned for use with the device of the present invention. The housings and control elements of the device of the present invention are described in conjunction with and shown in FIGS. 8A-8G.

With reference to FIGS. 1 and 2, there is seen a device, referenced generally 100, for applying a high velocity liquid-gas therapeutic stream to tissue for therapeutic treatment thereof. Alternatively, the velocity of the stream can be regulated to provide cleansing of the tissue. Device 100 includes a housing portion 102 having a generally tubular configuration, and having proximal and distal ends, referenced generally 104 and 106, respectively. A gas inlet port 108, and a liquid inlet port 110 are provided at proximal end 104, and a stream jet delivery nozzle arrangement 112 is provided at distal end 106.

In FIG. 2, there is shown, in schematic form, a therapeutic liquid inlet port 109 connecting pressurized therapeutic liquid source 107 via flow control element 105 to liquid inlet port 110 to allow production of a mixed flow of therapeutic liquid. It should be noted that the present arrangement producing one mixed therapeutic liquid flow is only shown by way of example, and that multiple therapeutic liquid flows, as well as control of the time of application of different therapeutic liquid flows are also contemplated as being part of the discussion herein.

Referring now to FIGS. 3 and 4 in conjunction with FIG. 2, there are schematic and graphical cross-sectional views of nozzle arrangement 112 of device 100. Nozzle arrangement 112 includes a gas discharge nozzle 114 and disposed generally concentrically there-within, is a liquid discharge nozzle 116. Liquid inlet port 110 of FIG. 2 is connected in fluid flow communication with liquid discharge nozzle 116 by means of a liquid communication tube 118, disposed generally concentrically within tubular housing portion 102 as shown in FIGS. 2 and 3.

Pressurized gas supplied from a pressurized gas source (not shown) enters device 100 through gas inlet port 108 of FIG. 2, and passes along and within tubular housing portion 102 as indicated by arrows 134, so as to discharge through gas discharge nozzle 114. Gas discharge nozzle 114 can be configured having, in flow succession, a converging portion 120, a throat portion 122 and a diverging discharge portion 124. The pressurized gas discharging from nozzle 114, as indicated by arrows 126, undergoes a rapid and substantial reduction in pressure to atmospheric pressure and a substantial acceleration to a high velocity, within the range of subsonic to supersonic velocity and specifically to a supersonic velocity. Gas discharge nozzle 114 is configured such that the discharging gas has an average cone angle of less than 10 degrees; that is, providing a substantially parallel gas flow.

Liquid, including therapeutic substances, from one or more pressurized therapeutic liquid sources (not shown) enters device 100 through liquid inlet port 110 and passes, as indicated by arrow 132, through liquid communication tube 118. In turn, at distal end 106, therapeutic liquid is discharged through an opening 128 in the distal end of liquid discharge nozzle 116 into discharging flow 126 of gas, the therapeutic liquid flow being indicated by arrow 130.

It will be appreciated by persons skilled in the art that, as the pressurized discharging gas emerges 126 from gas discharge nozzle 114 into the atmosphere, it undergoes a rapid drop in pressure to atmospheric pressure. The sudden pressure drop results in a substantial acceleration of the velocity of the discharging gas flow that approximates or even exceeds the velocity of sound and results in the production of a shock wave. The effect of the shock wave is to atomize the therapeutic liquid discharging from liquid discharge nozzle 116 into the flow of gas as a stream of therapeutic liquid droplets 130, such that there is obtained a relatively narrow jet of therapeutic liquid droplets in a high velocity gas flow 126.

Further, by way of example, the proportion of liquid flow to gas flow is extremely low due to the relatively high gas pressure of about 100 pounds per square inch (“psi”) and low liquid pressure of about 2 psi, as well as the relatively large internal diameter of gas discharge nozzle 114, for example 0.5 mm, compared to a small internal diameter, for example 0.09 mm, of liquid discharge nozzle 116. Consequently, little liquid tends to accumulate at the site to be cleaned or treated with one or more therapeutic substances. Furthermore, the relatively high gas flow has the effect of dispersing any accumulated liquid. When using a jet utilizing only liquid for cleansing, the liquid tends to accumulate on the tissue surface resulting in formation of a virtually stagnant liquid boundary layer close to and in contact with the surface, thereby reducing the effectiveness of cleansing. The very thin to negligible layer of liquid produced on the tissue surface by the above described nozzle arrangement allows more efficient dosage of additional therapeutic substances to the tissue surface, including the possibility of subcutaneous application of the therapeutic substances.

Referring now to FIG. 5, there is seen a high velocity flow of therapeutic liquid droplets referenced 140 discharging, in a high velocity gas flow 126, from nozzle arrangement 112 against a tissue surface referenced 142 to be cleaned and/or treated with therapeutic substances. Device 100 can be held in the hand of a user by housing portion 102.

Referring now to FIG. 6, there is seen a flow of therapeutic liquid droplets 140 discharging, in a high velocity gas flow 126, from nozzle arrangement 112 of device 100 into a periodontal pocket 144 disposed between a gum 146 and a tooth wall 148. Device 100 is held in the hand of a user by housing portion 102. For example, this procedure is effective for cleansing periodontal pockets, subsequent to a dental descaling treatment, so as to remove plaque and calculus debris as well as bacteria and the toxins produced by the bacteria, which otherwise lead to mechanical irritation and inflammation of the gingiva. In certain embodiments, device 100 can be used to apply desired dental therapeutic substances, such as antibiotics or anesthetics to the dental pocket.

Referring now to FIG. 7, there is seen, according to an alternative construction of the above described device, a cross-sectional view of a device (not shown) having a housing portion 102 and a multiple nozzle arrangement, depicted by reference number 150. Nozzle arrangement 150 is configured having multiple gas discharge nozzles 152 and multiple therapeutic liquid discharge nozzles 154, disposed generally concentrically within each gas nozzle 152 and projecting there-beyond. Such multiple nozzle arrangement 150 facilitates increasing the rate of tissue cleaning. In certain embodiments, the device supports multiple therapeutic liquid flows, which may be individually controlled.

The foregoing examples of the related art and limitations related therewith are intended to be illustrative and not exclusive. Other limitations of the related art will become apparent to those of skill in the art upon a reading of the specification and a study of the figures.

### Definition

In the discussion herein below, the term “distal” refers to the position on the devices discussed herein furthest from the user that is the portion closest to the nozzle arrangement of the devices. The term “proximal” refers to the position on the devices closest to the user that is the portion furthest from the nozzle arrangement of the devices.

The terms “cleanse”, “cleaning” and variants thereof in the discussion herein below, refers to the removal of solid contaminants, such as fibers, dust, sand particles, and the like, as well as the removal of organic matter, such as pus, fats, and the like from the surface of tissue being cleaned and/or being treated with therapeutic substances. The term “cleanse” includes lavage of hollow organs of the body.

The term “tissue” as used herein can refer to either human or animal tissue.

### SUMMARY

The following embodiments and aspects thereof are described and illustrated in conjunction with systems, tools and methods which are meant to be exemplary and illustrative, not limiting in scope.

There is provided, in accordance with an embodiment, a device for administering a liquid therapeutic substance to tissue, including a housing having a liquid inlet port; a gas inlet port connected to a pressurized gas source; at least one therapeutic substance supply assembly mounted onto said housing, each therapeutic substance supply assembly including: at least one syringe dispenser connector configured for receiving a syringe dispenser containing a predefined quantity or concentration of the liquid therapeutic substance, each syringe dispenser in fluid flow communication with one of at least one valve projecting from and external to said assembly, each of the at least one valve is associated with a different syringe dispenser and user operable between an open and a closed position, the at least one valve is adapted for control of a continuous flow of the at least one liquid therapeutic substance during operation of the device, the syringe dispenser includes a container configured to store the liquid therapeutic substance and a piston configured to be pulled into the container as the liquid therapeutic substance is removed from the container under suction so as to flow through said at least one valve; and, at least one liquid conduit in liquid communication the liquid inlet port and said at least one syringe dispenser connector; a stream jet delivery nozzle arrangement in fluid flow communication with the gas inlet port and in fluid flow communication with the conduit, operative to deliver the liquid therapeutic substance in an elevated velocity flow of gas discharged from the delivery nozzle arrangement at a predetermined concentration.

In certain embodiments, the container includes a collapsible bag configured to store the liquid therapeutic substance, the collapsible bag configured to collapse when liquid is removed from the collapsible bag.

In certain embodiments, the further includes at least one therapeutic substance supply assembly mounted onto the housing, each therapeutic substance supply assembly configured for receiving at least one syringe dispense containing a predefined quantity or concentration of liquid therapeutic substance.

In certain embodiments, the liquid therapeutic substance inlet port is in fluid flow communication with the therapeutic substance supply assembly and also in fluid flow communication with the stream jet delivery nozzle arrangement.

In certain embodiments, the stream jet delivery nozzle arrangement includes: at least one gas discharge nozzle arranged to receive a flow of pressurized gas from the gas inlet port and configured to accelerate the flow of gas so as to discharge it at an elevated velocity and, at least one liquid discharge nozzle arranged to receive a flow of liquid therapeutic substance from a therapeutic substance supply assembly and operative to discharge the flow of therapeutic substance into the elevated velocity flow of gas, thereby accelerating the velocity of the discharged liquid therapeutic substance as a stream of accelerated therapeutic droplets and to discharge the stream of accelerated therapeutic droplets towards a tissue mass for treatment by the therapeutic substance.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 is a perspective view of a prior art device for administering therapeutic substances to tissue;

FIG. 2 is a schematic side view of the prior art device of FIG. 1;

FIGS. 3 and 4 are enlarged schematic and graphical representations, respectively, of a delivery nozzle arrangement of the prior art device seen in FIGS. 1 and 2;

FIG. 5 is a schematic view of a flow of stream droplets discharging from the prior art delivery nozzle arrangement as seen in FIG. 4 against a surface to which therapeutic substances are to be administered;

FIG. 6 is a schematic view of a flow of stream droplets discharging from the prior art delivery nozzle arrangement seen in FIG. 4 into a periodontal pocket;

FIG. 7 is a schematic view of a prior art nozzle arrangement having multiple gas and liquid discharge nozzles;

FIGS. 8A-8C are perspective, side and top views, respectively, of a device for administering therapeutic substances to tissue constructed and operative in accordance with an embodiment of the present invention;

FIGS. 8D-8E are perspective and side views, respectively, of another device for administering therapeutic substances to tissue, constructed and operative substantially in accordance with the embodiment of the present invention shown in FIGS. 8A-8C,

FIG. 8F is a cut away side view of the therapeutic substance supply assembly in FIGS. 8A-8E;

FIG. 8G is a side view of a second embodiment of the present invention; and,

FIG. 9 is a schematic view of a device for administering therapeutic substances to tissues having syringe dispensers in accordance with certain embodiments.

### DETAILED DESCRIPTION

Disclosed herein is a device for administering therapeutic substances to tissue by directing a liquid-gas stream of droplets containing one or more therapeutic substances, according to certain embodiments.

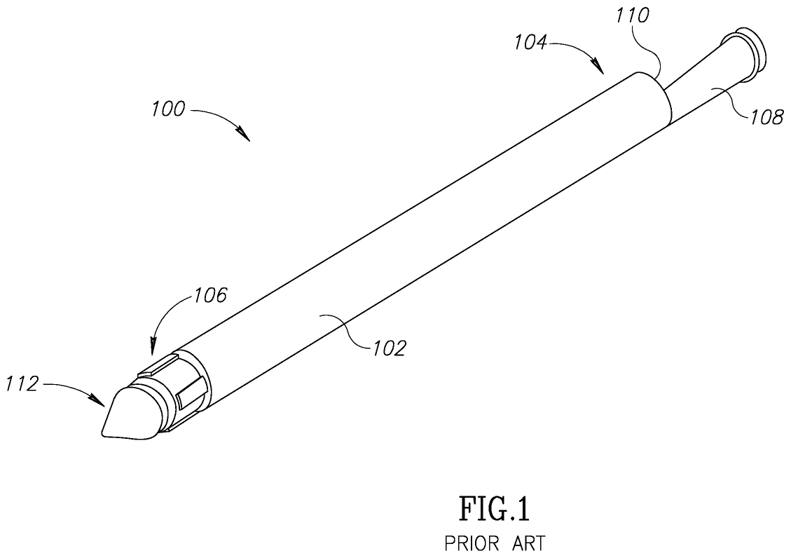

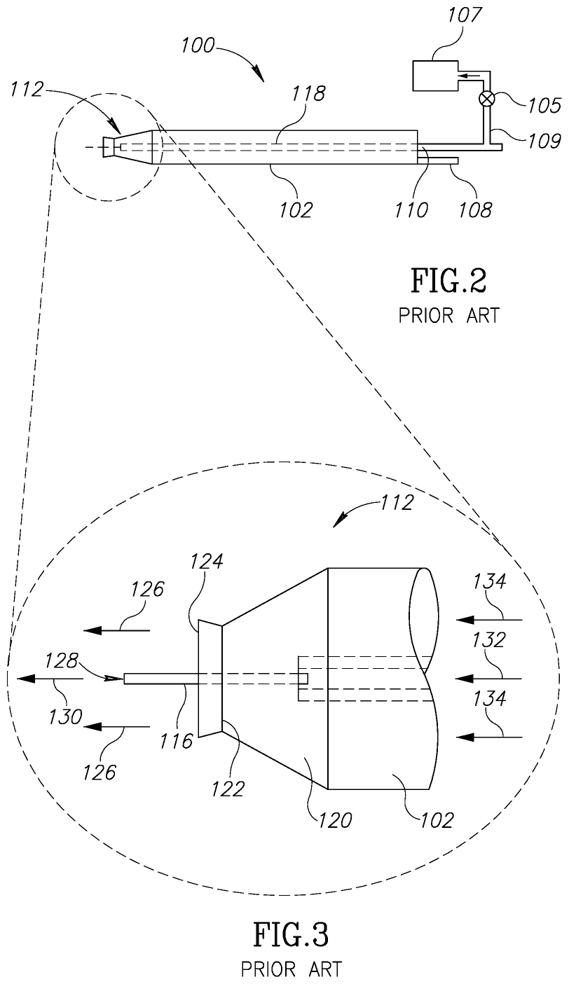

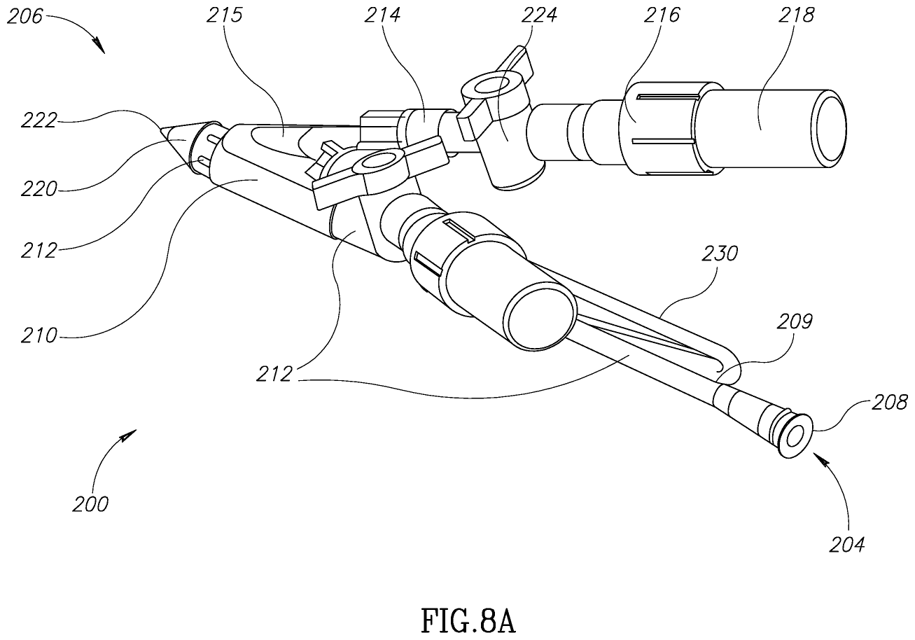

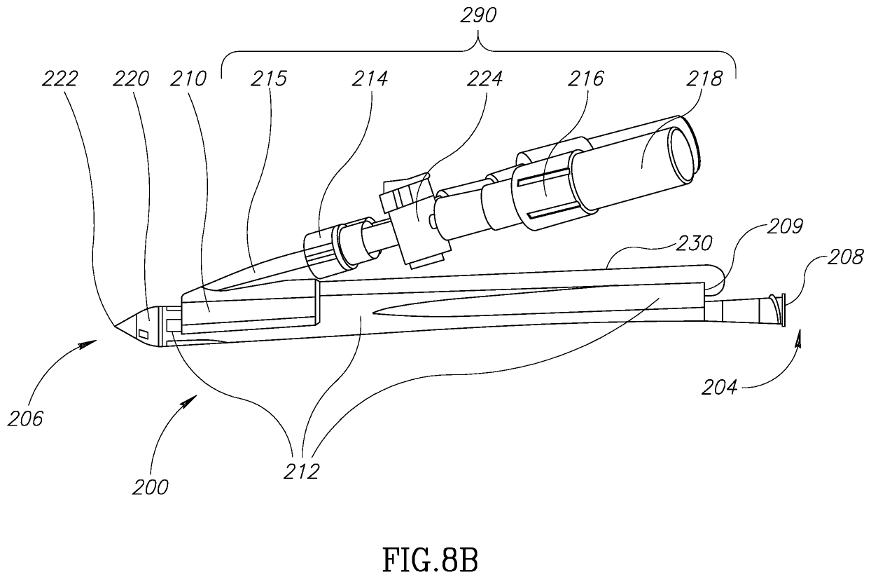

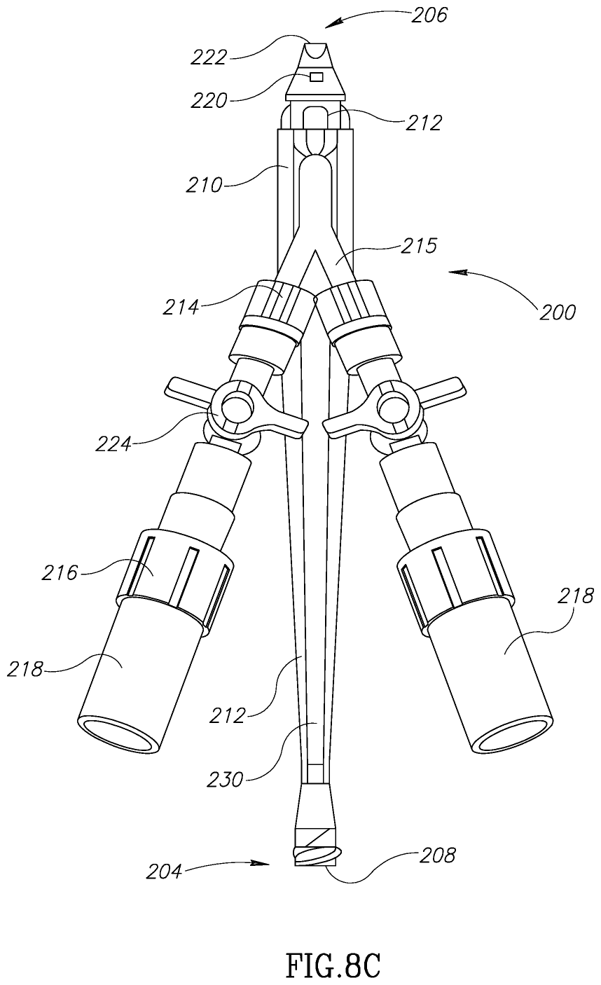

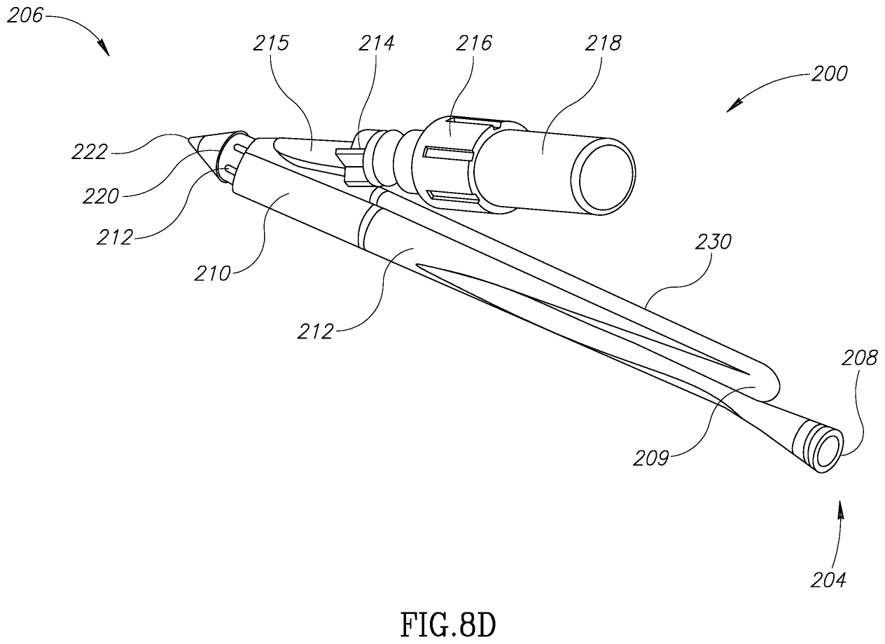

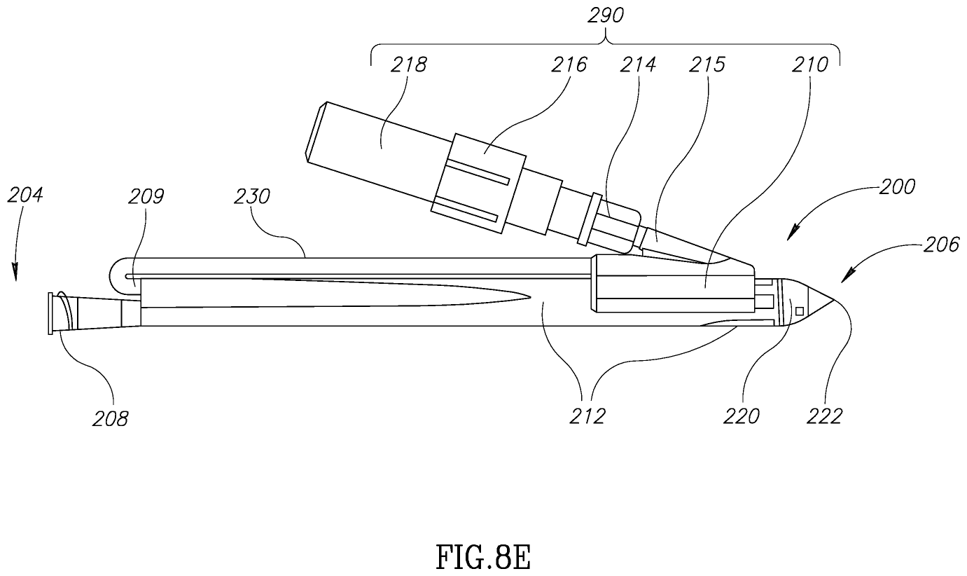

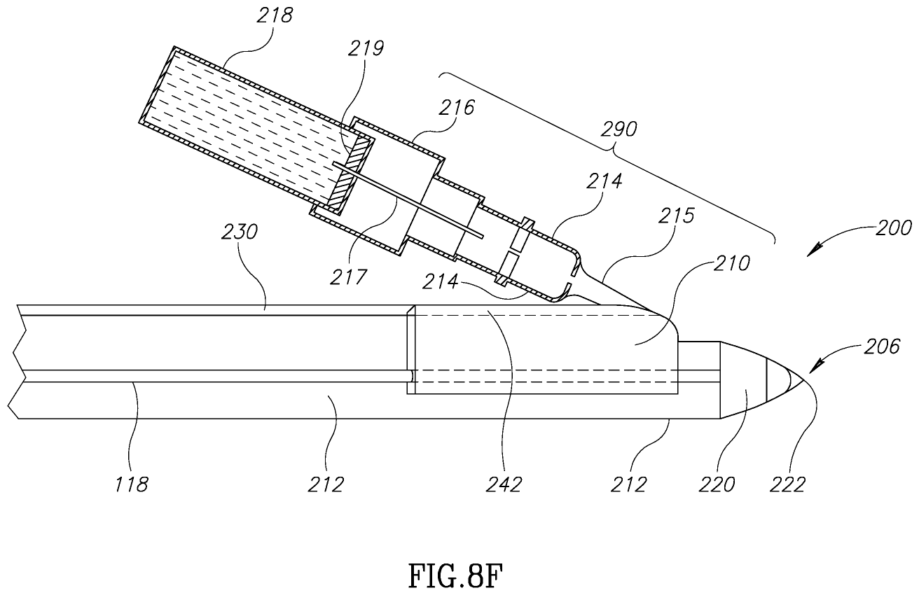

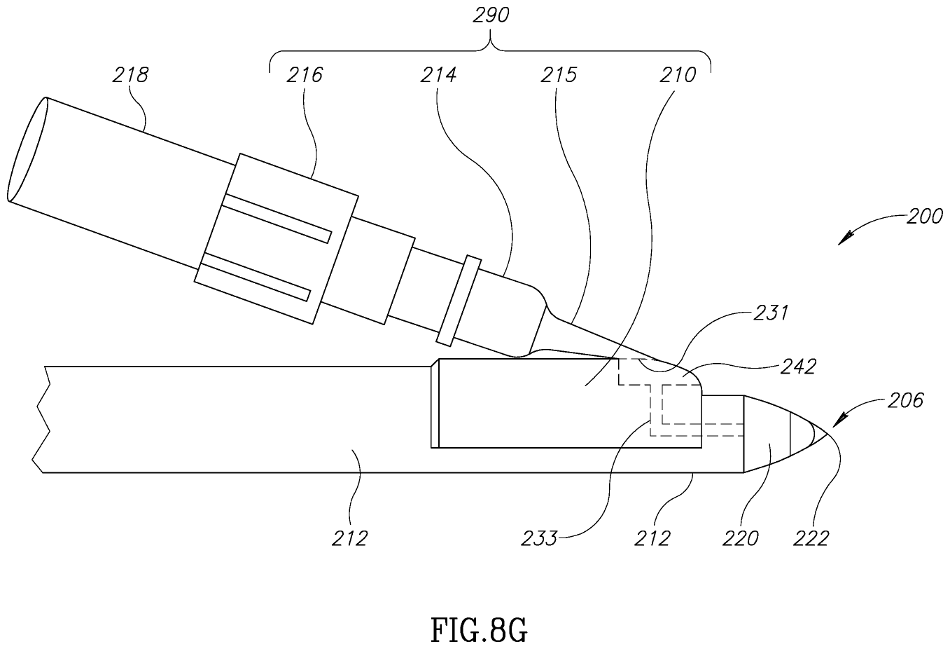

Referring now to FIGS. 8A-8C, there is seen, according to an embodiment of the present invention, a perspective, a side and a top view, respectively, of a device 200 configured to provide one or more (in the Figures one or two) therapeutic substances in predefined dosages and/or concentrations to a patient being treated using the present invention. Without intending to limit the invention, therapeutic substances which may be used include saline solutions, medicaments, nutrients, moisturizers or mixtures of any of these. The housing and control elements in FIGS. 8A-8C (as well as those in FIGS. 8D-8G discussed below) are different from the housing and control elements shown in FIGS. 1 and 2.

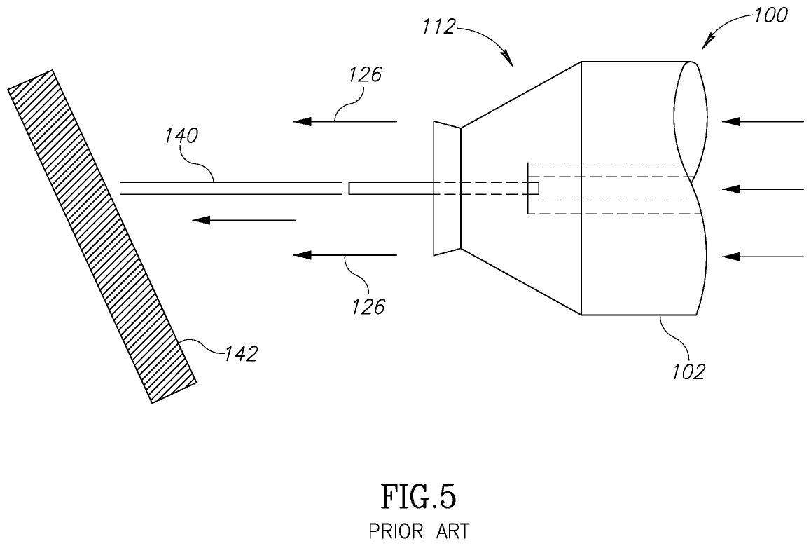

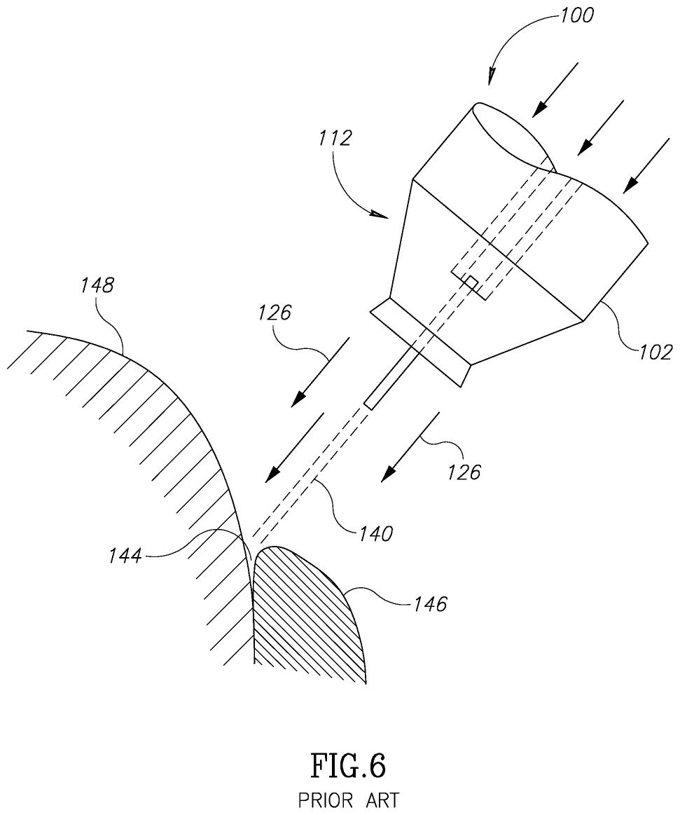

Nozzle arrangement 220, discharge nozzles 222 and hand piece housing portion 212 are constructed and configured substantially as described herein above and shown in FIGS. 1-7. Accordingly, description of these elements, their construction and their operation will not necessarily be repeated with respect to the embodiments of the invention presented and discussed in conjunction with FIGS. 8A-8G.

Two containers 218, such as, but without intending to limit the invention, bottles, vials or ampoules containing predefined dosages and/or concentrations of therapeutic liquid substances that are required in treating a patient, are positioned in container connectors 216. In certain embodiments, these containers 218 may be single-use containers. Container connectors 216 can be removably attachable and can be single-use connectors. Container connectors 216 can be connected by luer locks 214 to liquid conduits 215 that lead to assembly base 210.

In some embodiments, device 200 can include valves, such as stopcock valves 224, positioned between container connectors 216 and luer locks 214. In other embodiments (not shown), stopcock valves 224 can be positioned between luer locks 214 and liquid conduits 215. It should be appreciated by persons skilled in the art that valves other than stopcock valves may also be used.

While luer locks 214 generally are disclosed herein, it should readily be understood that other suitable connection fittings known to persons skilled in the art may also be used. In the claims, this element will generally be noted as “connection fittings” or “connection fitting”. Such designation is intended to include inter alia luer locks 214.

Assembly base 210, luer locks 214, stopcock valves 224, containers 218, container connectors 216, and liquid conduits 215 are typically, but without intending to limit the invention, made of rigid plastic. In certain embodiments, housing portion 212 can be formed of a rigid plastic. The exact plastics to be used for these elements are readily selectable by persons skilled in the art.

In certain embodiments, a side of assembly base 210 is disposed adjacent to device housing portion 212 and is shaped to conform to the adjacent side of housing portion 212. Assembly base 210 can be ultravioletly or ultrasonically bonded to housing portion 212. Alternatively, other methods of attachment known to persons skilled in the art suitable for use with plastics, such as adhesive gluing, can be used.

In certain embodiments, assembly base 210, luer lock 214, liquid conduit 215, stopcock valve 224 and container connector 216 can be constructed as an integral unit with handpiece housing portion 212 by using, for example, injection molding.

Container connectors 216, luer locks 214, liquid conduits 215, stopcock valves 224 and assembly base 210 collectively define, and will be herein referred to as a therapeutic substance supply assembly 290.

In some embodiments, such as the one discussed in conjunction with FIGS. 8D-8E below, there may be no need for stopcock valves. In such cases, the term “therapeutic substance supply assembly” 290 is previously defined but without the inclusion of stopcock or other valves.

In certain embodiments, a therapeutic substance supply assembly 290 is a structure attachable to a housing portion, such as element 212, including container connector 216, configured to receive a container, such as container 218. In certain embodiments, container 218 is in fluid flow communication with liquid discharge nozzles, such as discharge nozzles 222, of nozzle arrangement 220.

It should be understood that the specific embodiment of the therapeutic substance supply assemblies 290 shown in FIGS. 8A-8C and FIGS. 8D-8G are exemplary only. Other embodiments can be used if they perform the functions of the assembly 290 as discussed herein.

In certain embodiments, assembly base 210 is constructed and configured to fulfill two functions. First, it is configured to allow mounting of the therapeutic substance supply assembly 290 on housing portion 212. Second, assembly base 210 is formed with a conduit (242 in FIG. 8F), herein often denoted as an “assembly base conduit”, allowing fluid flow communication between therapeutic substance supply assembly 290 and liquid inlet port 209 (discussed below) via flexible tube 230.

In certain embodiments, container connector 216 can be a separate adaptor-like element screwable into, or otherwise removably positionable, in a conduit so that container 218, when positioned in connector 216, is in fluid flow communication with liquid conduits 215 and assembly base 210.

The therapeutic substances in containers 218 are conveyed through container connectors 216 either under gravity or as a result of the therapeutic substances in container 218 being provided under pressure. In certain embodiments, a puncturing element 217, as shown in FIG. 8F, can be present in container connector 216. Puncturing element 217 can puncture a cap 219 (FIG. 8F) of container 218, allowing the therapeutic substance to flow out of container 218 and ultimately into hand piece housing portion 212, as described below.

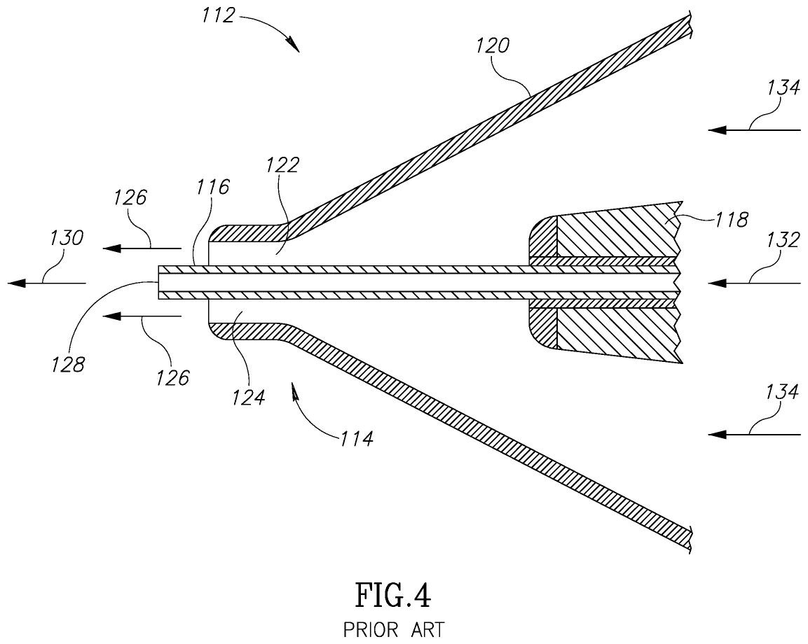

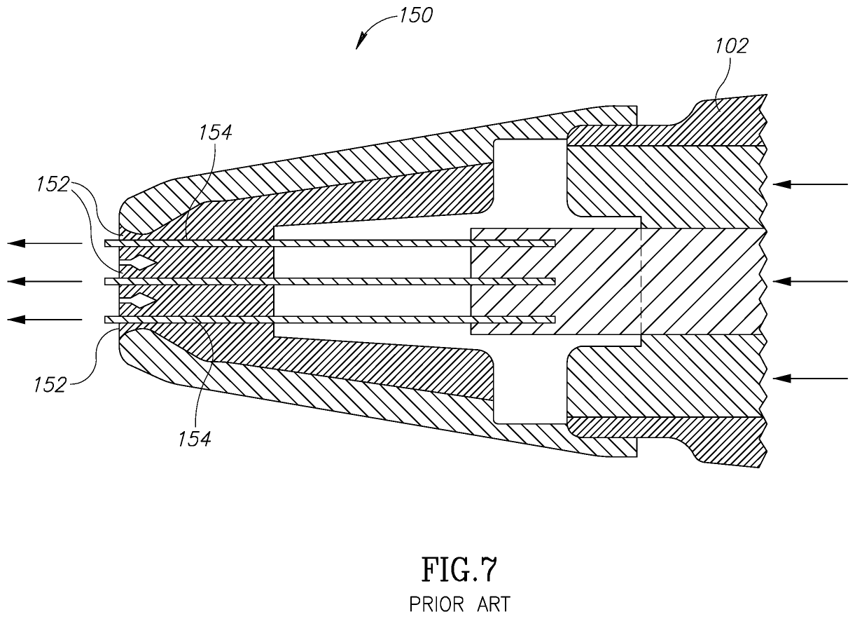

Stopcock valves 224 can be operated by the user to control flow of the therapeutic substance from containers 218 into housing portion 212. The operator may, by opening or closing stopcock valves 224, allow the therapeutic materials in one or both of therapeutic substance containers 218 to enter housing portion 212 and exit from nozzle arrangement 220 through liquid discharge nozzle(s) 222 (similar to elements 116 and 154 in, for example, FIGS. 4 and 7 respectively) at distal end 206 of device 200. The therapeutic liquid solution is then accelerated by pressurized gas exiting from gas discharge nozzles (similar to elements 114 and 152 in, for example, FIGS. 4 and 7, respectively) as discussed in conjunction with FIGS. 1-7.

The liquid therapeutic materials from containers 218 enter housing portion 212 through liquid inlet port 209. Liquid conduits 215 and the conduit formed in assembly base 210 (i.e. assembly base conduit—not shown) are in fluid flow communication with liquid inlet port 209. The liquid therapeutic materials flow from the conduit formed in assembly base 210 (i.e. the assembly base conduit 242 in FIG. 8F) through a flexible plastic tube 230 to port 209. From there, the liquid is transported either via flexible plastic tube 230 or liquid communication tube 118 (FIGS. 2 and 3) through housing portion 212 to discharge nozzle(s) 222 of nozzle arrangement 220.

A gas inlet port 208 and a liquid inlet port 209 are shown at proximal end 204 of device 200. Gas and liquid are introduced into device 200 through these ports from appropriate gas sources (not shown) and liquid sources (such as containers 218) as described above. The gas may be selected from air, oxygen, nitrogen and carbon dioxide but other non-reactive gases may also be used.

It should readily be understood by persons skilled in the art that the flow of a therapeutic substance from a container 218 positioned in a container connector 216 of a therapeutic substance supply assembly 290 to nozzle arrangement 220 can occur using any suitable fluid flow communication configuration.

According to certain embodiments, a side view schematically illustrated in FIG. 8G, to which reference is now made, shows housing portion 212 can be formed with an aperture 231 on its long tubular side intended for presentation to, and for fluid communication with, assembly base 210 of therapeutic substance supply assembly 290. An assembly base conduit 242 within assembly base 210, shown in FIG. 8G, can be configured to be in fluid flow communication and general registration with aperture 231 situated on housing portion 212.

A tube 233 equivalent to liquid communication tube 118 (the latter best seen in FIG. 2) of housing portion 212 is configured to be in fluid flow communication with therapeutic substance supply assembly 290 through aperture 231 and the assembly base conduit 242. At least one liquid discharge nozzle 222 substantially equivalent in construction and operation to liquid discharge nozzle 116 of FIGS. 2 and 4, receives the liquid therapeutic substance from the assembly base conduit 242 after it has passed through aperture 231 and liquid communication tube 233. The liquid is then discharged from liquid discharge nozzle(s) 222 in nozzle arrangement 220 (FIGS. 8A-8G).

In certain embodiments, no transmission tube 230 and no liquid inlet port 209 are required. In to certain embodiments, an adhesive, such as a silicon adhesive, which is used to connect therapeutic substance supply assembly 290 to housing portion 212 may also function as a sealant preventing loss of liquid during its transfer from containers 28 through assembly base 210 into housing portion 212, via aperture 231 and liquid communication tube 233.

Referring now to, FIGS. 8D and 8E showing a device 200 similar to device 200 in FIGS. 8A-8C but having only a single therapeutic substance supply assembly 290. Elements in 8D-8E are similar to therapeutic substance supplies 290 in FIGS. 8A-8C and are disclosed with the same reference number. All elements in FIGS. 8D-8E are constructed and operated as discussed in conjunction with FIGS. 8A-8C and therefore will not be described again. In FIGS. 8D-8E, no stopcock valve is present. In other embodiments of FIGS. 8D-8E, valves, such as, but not limited to, stopcock valves, may be added.

It should readily be evident to one skilled in the art that devices, such as device 200, may also be configured to operate with more than two therapeutic substance container connectors 216 and/or more than two therapeutic substance supply assemblies.

In certain embodiments, device 200 discussed in conjunction with FIGS. 8A-8G can be ergonomic and balanced for easier one-hand use by an operator.

In certain embodiments, device 200 can be used to apply the therapeutic droplet stream either topically or subcutaneously.

In certain embodiments, device 200 can be constructed to have a multiple nozzle configuration, similar to, for example, the one shown in and discussed hereinabove in conjunction with FIG. 7.

Because many therapeutic substances have a reduced shelf life after their original container has been opened, use of throw-away single-use therapeutic solution containers 218 obviates many difficulties readily apparent to persons skilled in the art. Moreover, since containers 218 to be used may be selected from among containers that may contain a wide variety of therapeutic substances each being manufactured at different quantities and/or concentrations, the use of such containers is an advantage. The positioning of therapeutic containers 218 directly on operating devices 200 allows for ease of use of devices 200 by reducing the need for restricting tubing and conduits. Devices 200 therefore are more easily adapted for single hand use by the user.

In certain embodiments, container 218 can be a collapsible bag, which collapses as the liquid therapeutic substance is sucked into device 200 due to a vacuum created in container 218 resulting from removal of the liquid therapeutic substance from container 218. In certain embodiments, container 218 can include a rigid housing for storing the collapsible bag containing the liquid therapeutic substances to prevent the collapsible bag from tearing.

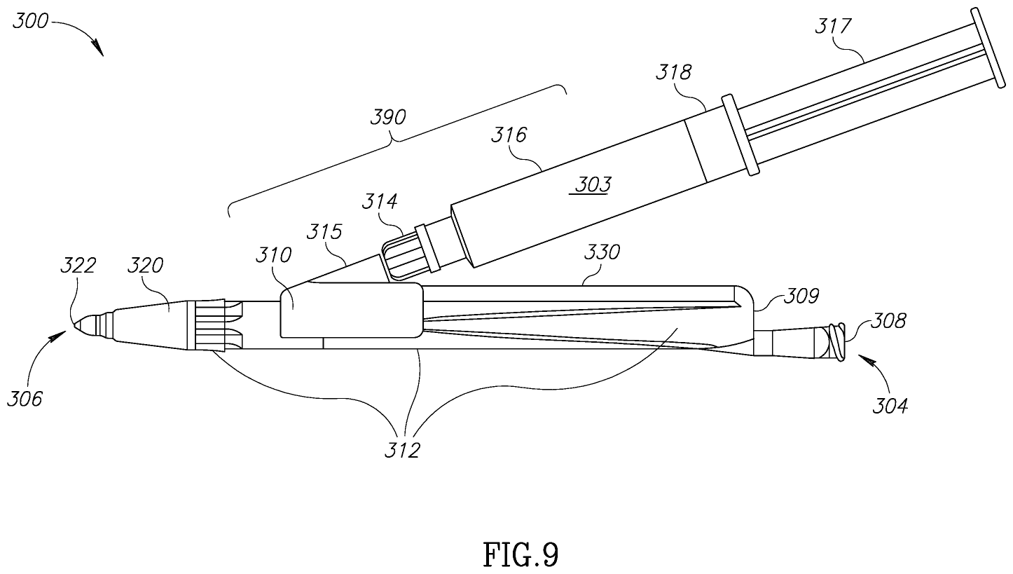

Referring now to FIG. 9 there is seen, according to an embodiment of the present invention, a view, of a device 300 configured to provide one or more therapeutic substances in predefined dosages and/or concentrations to a patient being treated using the present invention. Without intending to limit the invention, therapeutic substances which may be used include saline solutions, medicaments, nutrients, moisturizers or mixtures of any of these. The housing and control elements in FIG. 9 are different from the housing and control elements shown in FIGS. 1 and 2.

Nozzle arrangement 320, discharge nozzles 322 and hand piece housing portion 312 are constructed and configured substantially as described herein above and shown in FIGS. 1-7. Accordingly, description of these elements, their construction and their operation will not necessarily be repeated with respect to the embodiments of the invention presented and discussed in conjunction with FIG. 9.

A syringe dispenser 318 includes a container 303 configure to store predefined dosages and/or concentrations of therapeutic liquid substances that are required in treating a patient, are positioned in dispenser connectors 316. Syringe dispensers 318 can be single-use dispensers or multi-use dispensers. Dispenser connectors 3316 can be removably attachable and can be single-use connectors. Dispenser connectors 316 can be connected by luer locks 314 to liquid conduits 315 that lead to assembly base 310.

The therapeutic materials in syringe dispenser 318 enters housing portion 312 and exits from nozzle arrangement 320 through liquid discharge nozzle(s) 322 (similar to elements 116 and 154 in, for example, FIGS. 4 and 7 respectively) at distal end 306 of device 300. The therapeutic liquid solution is then accelerated by pressurized gas exiting from gas discharge nozzles (similar to elements 114 and 152 in, for example, FIGS. 4 and 7, respectively) as discussed previously in conjunction with FIGS. 1-7.

The liquid therapeutic materials from syringe dispense 318 enter housing portion 312 of device 300 through liquid inlet port 309. Liquid conduits 315 and the conduit formed in assembly base 310 (i.e. assembly base conduit—not shown) are in fluid flow communication with liquid inlet port 309. The liquid materials flow from the conduit formed in assembly base 310 through a flexible plastic tube 330 to inlet port 309. From there, the liquid therapeutic material is transported either via flexible plastic tube 330 or liquid communication tube 118 (FIGS. 2 and 3) through housing portion 312 to discharge nozzle(s) 322 of nozzle arrangement 320.

A gas inlet port 308 and liquid inlet port 309 are shown at the proximal end 304 of device 300. Gas and liquid are introduced into device 300 through these ports from appropriate gas sources (not shown) and liquid sources (such as syringe dispenser 318) as described above. The gas may be selected from air, oxygen, nitrogen and carbon dioxide but other non-reactive gases may also be used.

It should readily be understood by persons skilled in the art that the flow of a therapeutic substance from a syringe dispenser 318 positioned in a dispenser connector 316 of a therapeutic substance supply assembly 390 to nozzle arrangement 320 can occur using any suitable fluid flow communication.

Syringe dispenser 318 includes a piston 317 connected to container 303 enabling piston 317 to ingress and egress into container 303 to reduce the space within container 303. Piston 317 is configured to be pulled into container 303 as the therapeutic liquid substance is removed under suction from container 303. More specifically, as the therapeutic liquid substance flows out of container 303, a vacuum is generated within container 303, which pulls piston 317 into container to reduce the space within container 303 and prevents gas from entering container 303, which may contaminate the remaining therapeutic liquid substances.

In certain embodiments, an empty container 303 of syringe dispenser 318 can be replaced with a full container. For example, piston 317 can be disconnected from syringe dispenser 318 to enable quick replacement of an empty container and a new full container without disconnecting syringe dispenser 318 from dispenser connector 316.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A device for administering a liquid therapeutic substance to tissue, including:
a housing having a liquid inlet port;
a gas inlet port connected to a pressurized gas source;
at least one therapeutic substance supply assembly mounted onto said housing,
each therapeutic substance supply assembly including:
at least one syringe dispenser connector configured for receiving a syringe dispenser containing a predefined quantity or concentration of the liquid therapeutic substance, each syringe dispenser in fluid flow communication with one of at least one valve projecting from and external to said assembly, each of said at least one valve is associated with a different syringe dispenser and user operable between an open and a closed position, said at least one valve is adapted for control of a continuous flow of said at least one liquid therapeutic substance during operation of said device,
said syringe dispenser includes a container configured to store the liquid therapeutic substance and a piston configured to be pulled into the container as the liquid therapeutic substance is removed from said container under suction so as to flow through said at least one valve; and,
at least one liquid conduit in liquid communication said liquid inlet port and said at least one syringe dispenser connector;
a stream jet delivery nozzle arrangement in fluid flow communication with said gas inlet port and in fluid flow communication with said conduit, operative to deliver the liquid therapeutic substance in an elevated velocity flow of gas discharged from said delivery nozzle arrangement at a predetermined concentration.

2. A device according to claim 1, wherein said container includes a collapsible bag configured to store the liquid therapeutic substance, said collapsible bag configured to collapse when liquid is removed from said collapsible bag.

3. A device according to claim 1, further including at least one therapeutic substance supply assembly mounted onto said housing, each therapeutic substance supply assembly configured for receiving at least one syringe dispense containing a predefined quantity or concentration of liquid therapeutic substance.

4. A device according to claim 1, wherein said liquid therapeutic substance inlet port is in fluid flow communication with said therapeutic substance supply assembly and also in fluid flow communication with said stream jet delivery nozzle arrangement.

5. A device according to claim 1, wherein said stream jet delivery nozzle arrangement includes:
i) at least one gas discharge nozzle arranged to receive a flow of pressurized gas from said gas inlet port and configured to accelerate the flow of gas so as to discharge it at an elevated velocity; and,
ii) at least one liquid discharge nozzle arranged to receive a flow of liquid therapeutic substance from a therapeutic substance supply assembly and operative to discharge the flow of therapeutic substance into the elevated velocity flow of gas,
thereby accelerating the velocity of the discharged liquid therapeutic substance as a stream of accelerated therapeutic droplets and to discharge the stream of accelerated therapeutic droplets towards a tissue mass for treatment by the therapeutic substance
